(19) United States
(12) Patent Application Publication
(10) Pub. No.: US 2014/0088047 A1
(43) Pub. Date: Mar. 27, 2014

(54) USE OF LONG CHAIN POLYUNSATURATED FATTY ACID DERIVATIVES TO TREAT SICKLE CELL DISEASE

(71) Applicant: AKER BIOASSIST AS, Oslo (NO)

(72) Inventors: Nils Hoem, Oslo (NO); Elizabeth B. Vadas, Dorval (CA)

(73) Assignee: AKER BIOASSIST AS, Oslo (NO)

(21) Appl. No.: 14/035,206

(22) Filed: Sep. 24, 2013

Related U.S. Application Data
(60) Provisional application No. 61/704,856, filed on Sep. 24, 2012.
(60) Provisional application No. 61/739,263, filed on Dec. 19, 2012.
(60) Provisional application No. 61/775,021, filed on Mar. 8, 2013.

(51) Int. Cl. C07F 9/113; A61K 31/122; A23L 1/30; A61K 31/661; A61K 31/202; C07C 57/03; A61K 45/06

(52) U.S. Cl. CPC C07F 9/113; C07C 57/03; A61K 31/122; A61K 45/06; A61K 31/661; A61K 31/202; A23L 1/3008
USPC 514/120; 514/560

(57) ABSTRACT

The present invention relates to the use of long chain polyunsaturated fatty acids to treat diseases associated with red blood cells and cell membranes, and in particular to the use of derivatives of long chain fatty acids to treat sickle cell disease.

### CROSS-REFERENCE TO RELATED APPLICATIONS

This application claims priority to pending U.S. Provisional Patent Application No. 61/704,856, filed Sep. 24, 2012, and to pending U.S. Provisional Patent Application No. 61/739,263, filed Dec. 19, 2012, and to pending U.S. Provisional Patent Application No. 61/775,021, filed Mar. 8, 2013, the contents of which are incorporated by reference in their entireties.

### FIELD OF THE INVENTION

The present invention relates to the use of long chain polyunsaturated fatty acids to treat diseases associated with red blood cells and cell membranes, and in particular to the use of derivatives of long chain fatty acids to treat sickle cell disease.

### BACKGROUND OF THE INVENTION

Sickle cell disease (SCD) affects millions of people throughout the world and is particularly common among those whose ancestors came from sub-Saharan Africa; Spanish-speaking regions in the Western Hemisphere (South America, the Caribbean, and Central America); Saudi Arabia; India; and Mediterranean countries such as Turkey, Greece, and Italy.

In SCD, the red blood cells contain an abnormal form of hemoglobin (the protein that carries oxygen) that reduces the amount of oxygen in the cells, causing them to become crescent- or sickle-shaped. The sickle-shaped cells block and damage the smallest blood vessels in the spleen, kidneys, brain, bones, and other organs, reducing their oxygen supply. Because these deformed cells are fragile, they break up as they travel through blood vessels, causing severe anemia, blocked blood flow, organ damage and possibly death. SCD can also be extremely painful, especially for children who can exhibit severe chest pain and shortness of breath.

It is estimated that SCD affects 90,000 to 100,000 Americans and occurs among about 1 out of every 500 Black or African-American births. SCD occurs among about 1 out of every 36,000 Hispanic-American births. Sickle cell trait occurs among about 1 in 12 Blacks or African Americans. SCD is a major public health concern. From 1989 through 1993, an average of 75,000 hospitalizations due to SCD occurred in the United States, costing approximately $475 million.

Accordingly, what is needed in the art are safe and effective treatments for SCD, especially treatments that are safe for children afflicted with the disease.

### SUMMARY OF THE INVENTION

The present invention relates to the use of long chain polyunsaturated fatty acids to treat diseases associated with red blood cells and cell membranes, and in particular to the use of derivatives of long chain fatty acids to treat sickle cell disease.

The present invention relates to the use of long chain polyunsaturated fatty acids to treat diseases associated with red blood cells and cell membranes, and in particular to the use of derivatives of long chain fatty acids to treat sickle cell disease.

According, in some embodiments, the present invention provides methods for a) treating a disease or condition associated with abnormal red blood cells or cell membranes, b) improving the quality of life of an individual with sickle cell disease, or c) providing palliative for an individual with sickle cell disease, said methods comprising administering a composition comprising at least one compound selected from the group consisting of a phospholipid compound:

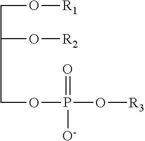

wherein at least one of R1 and R2 is a long chain polyunsaturated fatty acid moiety, the other of R1 and R2 is H or other organic moiety, and R3 is H or selected from a choline, ethanolamine, inositol and serine moiety,

a glyceride compound:

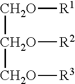

wherein at least one of either R1, R2, and R3 is a long chain polyunsaturated fatty acid moiety, and the other two of either R1, R2 and R3 may be H or a fatty acid moiety, including a long chain polyunsaturated fatty acid moiety,

esters and free fatty acids of long chain polyunsaturated fatty acids, and combinations thereof, for use in treating a disease or condition associated with abnormal red blood cells or cell membranes. In some embodiments, the composition comprises at least 30% long chain polyunsaturated fatty acid moieties on a w/w basis. In some embodiments, the composition comprises at least 40% long chain polyunsaturated fatty acid moieties on a w/w basis. In some embodiments, the composition comprises at least 60% long chain polyunsaturated fatty acid moieties on a w/w basis. In some embodiments, the composition comprises at least 80% long chain polyunsaturated fatty acid moieties on a w/w basis. In some embodiments, the long chain fatty acid moieties are selected from the group consisting of eicosapentaenoic acid, docosahexaenoic acid, and combinations thereof. In some embodiments, the composition comprises at least 10%, 20%, 30%, or 40% w/w of said phospholipid compounds. In some embodiments, the composition comprises at least 60% w/w of said phospholipid compounds. In some embodiments, the composition comprises at least 80% w/w of said phospholipid compounds. In some embodiments, the composition comprises a mixture of said phospholipid compounds, said mixture comprising at least 30% w/w long chain polyunsaturated fatty acid moieties. In some embodiments, the composition comprises a mixture of said phospholipid compounds, said mixture comprising at least 20% w/w long chain polyunsaturated fatty acid moieties. In some embodiments, the composition comprises a mixture of said phospholipid compounds, said mixture comprising at least 40% w/w long chain polyunsaturated fatty acid moieties. In some embodiments, the composition comprises a mixture of said phospholipid compounds, said mixture comprising at least 50% w/w long chain polyunsaturated fatty acid moieties. In some embodiments, the long chain polyunsaturated fatty acid moieties are selected from the group consisting of eicosapentaenoic acid, docosahexaenoic acid, and combinations thereof. In some embodiments, greater than 90% w/w of said long chain polyunsaturated fatty acid moieties selected from the group consisting of eicosapentaenoic acid, docosahexaenoic acid, and combinations thereof are bound at position R2 of said phospholipid compound. In some embodiments, the eicosapentaenoic acid and said docosahexaenoic acid are present in a ratio of eicosapentaenoic acid:docosahexaenoic acid of from about 1:1 to about 3:1. In some embodiments, the composition comprises a mixture of said phospholipid compounds, said mixture comprising at least 80% of said phospholipid compounds having a choline moiety at position R3.

In some embodiments, the composition comprises at least 10% ethyl esters of said long chain polyunsaturated fatty acids. In some embodiments, the composition comprises at least 30% ethyl esters of said long chain polyunsaturated fatty acids. In some embodiments, the composition comprises at least 50% ethyl esters of said long chain polyunsaturated fatty acids. In some embodiments, the esters of long chain polyunsaturated fatty acid comprise a long chain polyunsaturated fatty acid moiety selected from the group consisting of eicosapentaenoic acid, docosahexaenoic acid, and combinations thereof.

In some embodiments, the composition comprises a mixture of said glyceride compounds, said mixture comprising at least 30% w/w long chain polyunsaturated fatty acid moieties. In some embodiments, the composition comprises a mixture of said glyceride compounds, said mixture comprising at least 40% w/w long chain polyunsaturated fatty acid moieties. In some embodiments, the composition comprises a mixture of said glyceride compounds, said mixture comprising at least 50% w/w long chain polyunsaturated fatty acid moieties. In some embodiments, the long chain polyunsaturated fatty acid moieties are selected from the group consisting of eicosapentaenoic acid, docosahexaenoic acid, and combinations thereof.

In some embodiments, the composition comprises astaxanthin. In some embodiments, the composition comprises at least a second antioxidant. In some embodiments, the composition is partially or totally derived from krill.

In some embodiments, the composition is administered in a formulation selected from the group consisting of a capsule, a tablet, a liquid, a powder, an emulsion, a dietary supplement, a nutritional supplement, a beverage and a functional food. In some embodiments, the composition is administered by a route selected from the group consisting of oral administration and intravenous administration. In some embodiments, the composition is administered in a daily dose of from about 0.1 to about 3 grams.

In some embodiments, the composition is administered to a subject selected from the group consisting of humans, non-human primates, domestic raised or farmed animals, and companion animals. In some embodiments, the disease or condition associated with abnormal red blood cells or cell membranes is selected from the group consisting of sickle cell anemia, sickle cell disease, sickle cell trait, thalassemia, hemaglobinopathies, splenomegaly, presence of acanthocytes, presence of codocytes, presence of echinocytes, presence of burr cells, presence of elliptocytes, presence of ovalocytes, presence of spherocytes, presence of stomatocytes, presence of degmacytes, and combinations thereof.

In some embodiments, a second active agent is coadministered with the LC-PUFA composition. In some embodiments, the second active agent is selected from the group consisting of hydroxyurea, chelators, antibiotics, pain relievers, NSAIDs, niprisan, piracetam, selectin inhibitors, ion channel blockers, and DNA methyltransferase inhibitors.

In some embodiments, the present invention provides a composition comprising an LC-PUFA composition as described above and a second active agent. In some embodiments, the second active agent is selected from the group consisting of hydroxyurea, chelators, antibiotics, pain relievers, NSAIDs, niprisan, piracetam, selectin inhibitors, ion channel blockers, and DNA methyltransferase inhibitors. In some embodiments, the composition comprises effective amounts of said LC-PUFA compositions and said second active agent. In some embodiments, the effective amounts are sufficient to prevent, alleviate, or alter one or more symptoms or conditions associated with a disease associated with red blood cells and cell membranes, especially sickle cell disease.

In some embodiments, the present invention provides for use of a composition comprising a mixture of phospholipid compounds:

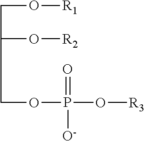

wherein at least one of R1 and R2 is a long chain polyunsaturated fatty acid moiety, the other of R1 and R2 is H or an organic moiety, and R3 is H or selected from a choline, ethanolamine, inositol and serine moiety, wherein greater than 90% w/w of said long chain polyunsaturated fatty acid moieties are selected from the group consisting of eicosapentaenoic acid, docosahexaenoic acid, and combinations thereof and are bound at position R2 of said phospholipid compounds, and wherein at least 80% of said phospholipid compounds have a choline moiety at position R3, said composition further characterized in comprising greater than about 10% w/w of said phospholipid compounds and optionally at least one of: a mixture of glyceride compounds:

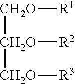

wherein at least one of either R1, R2, and R3 is a long chain polyunsaturated fatty acid moiety, and the other two of either R1, R2 and R3 may be H or a fatty acid moiety, wherein said long chain polyunsaturated fatty acid moieties are selected from the group consisting of eicosapentaenoic acid, docosahexaenoic acid, and combinations thereof, and a mixture of esters of long chain polyunsaturated fatty acids, wherein said long chain polyunsaturated fatty acid moieties are selected from the group consisting of eicosapentaenoic acid, docosahexaenoic acid, and combinations thereof, for a) treating a disease or condition associated with abnormal red blood cells or cell membranes, b) improving the quality of life of an individual with sickle cell disease, or c) providing palliative for an individual with sickle cell disease.

### DESCRIPTION OF THE FIGURES

FIGS. 1a and b are graphs showing a trend of positive effects on quality of life as measured by questionnaire. Patients evaluated quality of life by scoring 9 statements from 1-4 (e.g. “It is hard for me to breathe”, “It is hard for me to walk.” Scores for the statements were summarized for all subjects at each visit (2 subjects excluded due to withdrawal). There was a more pronounced decrease in scores in subjects with higher pain at Day 1.

FIG. 2 is a graph demonstrating that the reduction in pain scores increases over a 12 week period.

FIG. 3 is a graph demonstrating that variability in reduction in pain is less in the under 12 age group.

FIG. 4 is a graph showing that the reduction in pain effect is greater where the pain score is initially high.

FIGS. 5a and b are graphs showing that are no changes in bilirubin and hemoglobin.

### DEFINITIONS

As used herein, “phospholipid” refers to an organic compound having the following general structure:

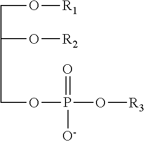

wherein R1 is a fatty acid residue or —H, R2 is a fatty acid residue or —H, and R3 is a —H or a phospholipid headgroup moiety such as a choline (HOCH₂CH₂N⁺(CH₃)₃OH⁻) moiety, ethanolamine (HOCH₂CH₂NH₂) moiety, serine moiety, inositol moiety such as cyclohexane polyol inositol, and derivatives thereof. Preferably, R1 and R2 cannot simultaneously be —H. When R3 is an —H, the compound is a diacylglycerophosphate, while when R3 is a nitrogen-containing compound, the compound is a phosphatide such as lecithin, cephalin, phosphatidyl serine or plasmalogen.

An “ether phospholipid” as used herein refers to a phospholipid having an ether bond at position 1 the glycerol backbone. Examples of ether phospholipids include, but are not limited to, alkylacylphosphatidylcholine (AAPC), lyso-alkylacylphosphatidylcholine (LAAPC), and alkylacylphosphatidylethanolamine (AAPE). A “non-ether phospholipid” is a phospholipid that does not have an ether bond at position 1 of the glycerol backbone.

As used herein, the term “long chain polyunsaturated fatty acid” refers to a fatty acid having 20 or more carbons and which is unsaturated at two or more bonds.

As used herein, the term omega-3 fatty acid refers to polyunsaturated fatty acids that have the final double bond in the hydrocarbon chain between the third and fourth carbon atoms from the methyl end of the molecule. Non-limiting examples of omega-3 fatty acids include, 5,8,11,14,17-eicosapentaenoic acid (EPA), 4,7,10,13,16,19-docosahexanoic acid (DHA) and 7,10,13,16,19-docosapentanoic acid (DPA).

As used herein, the term “moiety” when used in reference to a fatty acid refers to the portion of the fatty acid bound to another molecule via a bond, such as an ester or ether linkage to for example, a glyceride or phosphoglyceride molecule.

As used herein, the term “physiologically acceptable carrier” refers to any carrier or excipient commonly used with oily pharmaceuticals. Such carriers or excipients include, but are not limited to, oils, starch, sucrose and lactose.

As used herein, the term “oral delivery vehicle” refers to any means of delivering a pharmaceutical orally, including, but not limited to, capsules, pills, tablets and syrups.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

As used herein, the term “functional food” refers to a food product to which a biologically active supplement has been added.

As used herein, the term “infant food” refers to a food product formulated for an infant such as formula.

As used herein, the term “elderly food” refers to a food product formulated for persons of advanced age.

As used herein, the term “pregnancy food” refers to a food product formulated for pregnant women.

As used herein, the term “nutritional supplement” refers to a food product formulated as a dietary or nutritional supplement to be used as part of a diet.

### DETAILED DESCRIPTION OF THE INVENTION

The present invention relates to the use of long chain polyunsaturated fatty acids (LC-PUFA) to treat diseases associated with red blood cells and cell membranes, and in particular to the use of derivatives of long chain fatty acids to treat sickle cell disease.

Sickle cell anemia is characterized by a gene variant produces an abnormally shaped variant of hemoglobin. This molecule causes the typical “sickle” shape of the erythrocyte. Apart from the abnormal appearance, the erythrocyte membrane also becomes less plastic and flexible and thus its flow through the finest capillaries is severely hampered. The abnormal shape will also influence other physiological processes that are dependent on intact erythrocyte membrane fluidity and plasticity. In some embodiments, the compositions of the present invention reduce erythrocyte membrane stiffness and increase plasticity by increasing the relative amount of long chain polyunsaturated fatty acids in the erythrocyte membrane. In some preferred embodiments, this is achieved by supplementing/treating the patients (over time and probably starting at as early an age possible, for example at 1, 2, 3, 4, 5, 6, 7, 8, 9, or 10 years of age) with long chain polyunsaturated fatty acids (e.g., EPA and DHA), preferentially in their phospholipid form. The compositions of the present invention unexpectedly enhance the availability of the long chain polyunsaturated fatty acids to the erythrocyte forming tissues, namely red bone marrow and to a certain extent the spleen and the liver.

Accordingly, in some embodiments, the present invention utilizes compositions comprising one or more LC-PUFAs or LC-PUFA derivatives to treat diseases associated with red blood cells and cell membranes such as sickle cell disease.

In some embodiments, the LC-PUFA derivative is a phospholipid compound. Suitable phospholipid compounds include, but are not limited to, those described by the following structure:

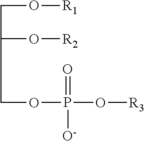

wherein at least one of R1 and R2 is a LC-PUFA moiety, the other of R1 and R2 is H or other organic moiety, and R3 is H or a phospholipid headgroup moiety, e.g., choline, ethanolamine, inositol or serine moiety. In some preferred embodiments, R2 is a LC-PUFA moiety and R1 is OH or other organic moiety.

In some embodiments, the LC-PUFA moiety is preferably an omega-3 fatty acid moiety selected from the group consisting of Eicosatrienoic acid (ETE; 20:3 (n-3); all-cis-11,14,17-eicosatrienoic acid); Eicosatetraenoic acid (ETA; 20:4 (n-3); all-cis-8,11,14,17-eicosatetraenoic acid); Eicosapentaenoic acid (EPA; 20:5 (n-3); all-cis-5,8,11,14,17-eicosapentaenoic acid); Heneicosapentaenoic acid (HPA; 21:5 (n-3); all-cis-6,9,12,15,18-heneicosapentaenoic acid); Docosapentaenoic acid (DPA; 22:5 (n-3); all-cis-7,10,13,16,19-docosapentaenoic acid; Docosahexaenoic acid (DHA; 22:6 (n-3); all-cis-4,7,10,13,16,19-docosahexaenoic acid); Tetracosapentaenoic acid (24:5 (n-3); all-cis-9,12,15,18,21-tetracosapentaenoic acid; and Tetracosahexaenoic acid (24:6 (n-3) all-cis-6,9,12,15,18,21-tetracosahexaenoic acid). In some embodiments, the LC-PUFA moiety is preferably an omega-6 fatty acid moiety selected from the group consisting of Eicosadienoic acid (20:2 (n-6); all-cis-11,14-eicosadienoic acid); Dihomo-gamma-linolenic acid (DGLA; 20:3 (n-6) all-cis-8,11,14-eicosatrienoic acid; Arachidonic acid (AA; 20:4 (n-6); all-cis-5,8,11,14-eicosatetraenoic acid); Docosadienoic acid (22:2 (n-6); all-cis-13,16-docosadienoic acid); Adrenic acid (22:4 (n-6) all-cis-7,10,13,16-docosatetraenoic acid; Docosapentaenoic acid (22:5 (n-6); all-cis-4,7,10,13,16-docosapentaenoic acid); Tetracosatetraenoic acid (24:4 (n-6); all-cis-9,12,15,18 tetracosatetraenoic acid); and Tetracosapentaenoic acid (24:5 (n-6); all-cis-6,9,12,15,18-tetracosapentaenoic acid). In some embodiments, the LC-PUFA moiety is preferably an omega-9 fatty acid moiety such as mead acid (20:3 (n-9); 5,8,11-eicosatrienoic acid). In some embodiments, the LC-PUFA moiety is bound through an ester bond at the R1 or R2 position (to provide an acylphospholipid), while in other embodiments, the LC-PUFA moiety is bound through an ether bond or vinyl ether bond (to provide an ether phospholipid, alkylacylphospholipid, or alkenylacylphospholipid). Moreover, LC-PUFA moieties may be conjugated, hydroxylated, epoxidated or hydroxyepoxidated acyl residues. In embodiments where at least one of R1 and R2 is a LC-PUFA moiety, the other of R1 and R2 may be any organic moiety which can be bound to the R1 and R2 positions through a suitable chemical bond. In some embodiments, the moiety is —H, providing a lysophospholipid with a LC-PUFA at either the R1 or R2 position. In other embodiments, the organic moiety is a fatty acid moiety bound to the R1 or R2 position via an ester, ether or vinyl ether bond. Exemplary fatty acids moieties include, but are not limited to, the omega-3, omega-6 and omega-9 moieties described above, as well as decanoic acid (10:0), undecanoic acid (11:0), 10-undecanoic acid (11:1), lauric acid (12:0), cis-5-dodecanoic acid (12:1), tridecanoic acid (13:0), myristic acid (14:0), myristoleic acid (cis-9-tetradecenoic acid, 14:1), pentadecanoic acid (15:0), palmitic acid (16:0), palmitoleic acid (cis-9-hexadecenoic acid, 16:1), heptadecanoic acid (17:1), stearic acid (18:0), elaidic acid (trans-9-octadecenoic acid, 18:1), oleic acid (cis-9-octadecanoic acid, 18:1), nonadecanoic acid (19:0), eicosanoic acid (20:0), cis-11-eicosenoic acid (20:1), 11,14-eicosadienoic acid (20:2), heneicosanoic acid (21:0), docosanoic acid (22:0), erucic acid (cis-13-docosenoic acid, 22:1), tricosanoic acid (23:0), tetracosanoic acid (24:0), nervonic acid (24:1), pentacosanoic acid (25:0), hexacosanoic acid (26:0), heptacosanoic acid (27:0), octacosanoic acid (28:0), nonacosanoic acid (29:0), triacosanoic acid (30:0), vaccenic acid (t-11-octadecenoic acid, 18:1), tariric acid (octadec-6-ynoic acid, 18:1), and ricinoleic acid (12-hydroxyoctadec-cis-9-enoic acid, 18:1). In some embodiments, fatty acids moieties may be conjugated, hydroxylated, epoxidated or hydroxyepoxidated acyl residues.

In some embodiments, compositions comprising the phospholipid compounds described above are utilized. In some embodiments, the compositions comprise a phospholipid fraction comprising a mixture of two or more of the phospholipid compounds described above. In some embodiments, the fatty acid content of the phospholipid fraction is from about 1% to about 99% LC-PUFA on a weight/weight basis (w/w; calculated as the weight of LC-PUFA in the phospholipid fraction divided by the total weight of fatty acids in the phospholipid fraction) or molar ratio basis (moles of LC-PUFA in the composition expressed as a percentage of the moles total fatty acids), 10% to 40% LC-PUFA w/w or molar ratio, 20% to 40% LC-PUFA w/w or molar ratio, 20% to 50% LC-PUFA w/w or molar ratio, 40% to 60% LC-PUFA w/w or molar ratio, 40% to 99% LC-PUFA w/w or molar ratio, 60% to 99% LC-PUFA w/w or molar ratio, or 80% to 99% LC-PUFA w/w or molar ratio. The w/w % may preferably be determined by an analytical method selected from the group consisting of gas chromatography (GC), high performance liquid chromatography (HPLC), GC-mass spectrometry (GC-MS), nuclear magnetic resonance (NMR) or other suitable methods as is known in the art. In some preferred embodiments, the LC-PUFA moieties are preferably selected from DHA, EPA and combinations thereof. In some embodiments, the LC-PUFA moieties are distributed between the R1 and R2 positions. In other embodiments, more than 90% w/w of the LC-PUFA moieties, preferably more than 95% w/w of the LC-PUFA moieties, and most preferably more than about 98% w/w of the LC-PUFAs are distributed at the R2 position. In some preferred embodiments, the LC-PUFA moieties are greater than 50%, 60%, 70%, 80%, 90% or 95% w/w EPA and/or DHA. In some embodiments, the ratio of EPA to DPA is from about 10:1 to 1:10, 3:1 to 1:3, 2:1 to 1:1, 5:1 to 1:1, 3:1 to 1:1, 1:1 to 1:3, or 1:1 to 1:5 on a molar basis. In some preferred embodiments, the composition preferably comprises greater than about 80%, 90%, or 95% phospholipid compounds w/w having a choline moiety at R3. In some embodiments, the compositions comprise greater than about 40%, 50%, 60%, 70%, 80%, 90% or 95% phospholipids w/w.

The phospholipid compounds of the present invention may be provided from a variety of sources. In some embodiments, the phospholipids are from a natural source, for example krill, herring, herring roe, copepods or other suitable sources. A suitable krill oil is described in WO/2008/117602, the entire contents of which are incorporated herein by reference. A suitable phospholipid concentrate produced from krill oil is described in U.S. Pat. Publ. 20110160161, the entire contents of which are incorporated herein by reference. In some embodiments, the present invention utilizes a krill oil composition, preferably a Euphausia superba krill oil composition, comprising from about 40% to about 60% w/w phospholipids, preferably from about 45% to 55% w/w phospholipids and from about 100 mg/kg astaxanthin to about 2500 mg/kg astaxanthin. In some preferred embodiments, the krill oil compositions of the present invention comprise from about 1%, 2%, 3% or 4% to about 8%, 10%, 12% or 15% w/w ether phospholipids or greater than about 4%, 5%, 6%, 7%, 8%, 9% or 10% ether phospholipids. In some embodiments the ether phospholipids are preferably alkylacylphosphatidylcholine, lyso-alkylacylphosphatidylcholine, alkylacylphosphatidyl-ethanolamine or combinations thereof. In some embodiments, the krill oil compositions comprise from about 1%, 2%, 3% or 4% to about 8%, 10%, 12% or 15% w/w ether phospholipids and from about 30%, 33%, 40%, 42%, 45%, 48%, 50%, 52%, 54%, 55% 56%, 58% to about 60% non-ether phospholipids so that the total amount of phospholipids (both ether and non-ether phospholipids) ranges from about 40% to about 60%. One of skill in the art will recognize that the range of 40% to 60% total phospholipids, as well as the other ranges of ether and non-ether phospholipids, can include other values not specifically listed within the range. In other embodiments, the phospholipid compounds may be produced by synthetic processes. Suitable synthetic processes are described, for example, in WO/2006/054183, the entire content of which are incorporated herein by reference.

In some embodiments, the LC-PUFA derivative is a glyceride compound. Suitable glyceride compounds include, but are not limited to, those described by the following structure:

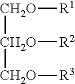

wherein at least one of either R1, R2, and R3 is a LC-PUFA moiety, and the other two of either R1, R2 and R3 may H or a fatty acid moiety, including a LC-PUFA moiety. In some embodiments, compositions comprising the glyceride compounds are utilized. In some embodiments, the compositions comprise a glyceride fraction comprising a mixture of two or more of the glyceride compounds described above. Preferred LC-PUFAs and fatty acids are described above in the description of the phospholipid compounds and compositions. In some embodiments, the fatty acid content of the glyceride fraction is from about 1% to about 99% LC-PUFA on a weight/weight basis (w/w; calculated as the weight of LC-PUFA in the glyceride fraction divided by the total weight of fatty acids in the glyceride fraction) or molar ratio basis (moles of LC-PUFA in the composition expressed as a percentage of moles total fatty acids), 10% to 40% LC-PUFA w/w or molar ratio, 20% to 40% LC-PUFA w/w or molar ratio, 20% to 50% LC-PUFA w/w or molar ratio, 40% to 60% LC-PUFA w/w or molar ratio, 40% to 99% LC-PUFA w/w or molar ratio, 60% to 99% LC-PUFA w/w or molar ratio, or 80% to 99% LC-PUFA w/w or molar ratio. The w/w % may preferably be determined by an analytical method selected from the group consisting of gas chromatography (GC), high performance liquid chromatography (HPLC), GC-mass spectrometry (GC-MS), nuclear magnetic resonance (NMR) or other suitable methods as is known in the art. In some preferred embodiments, the LC-PUFA moieties are preferably selected from DHA, EPA and combinations thereof. In some preferred embodiments, the LC-PUFA moieties are greater than 50%, 60%, 70%, 80%, 90% or 95% w/w EPA and/or DHA. In some embodiments, the ratio of EPA to DPA is from about 10:1 to 1:10, 3:1 to 1:3, 5:1 to 1:1, 3:1 to 1:1, 2:1 to 1:1, 1:1 to 1:3, or 1:1 to 1:5 on a molar basis. In some embodiments, the compositions comprise greater than about 40%, 50%, 60%, 70%, 80%, 90% or 95% glycerides w/w.

The glyceride compounds and compositions of the present invention may be provided from a variety of sources. In some embodiments, the glyceride compositions provided as a marine oil. Suitable marine oils include, but are not limited to, krill oil, tuna oil, herring oil, menhaden oil, cod liver oil and algae oil. A suitable krill oil is described in WO/2008/117602, the entire contents of which are incorporated herein by reference. The glycerides may also be produced synthetically. For example, it is known in the art that fish oils with concentrated amounts of desired LC-PUFA moieties such as EPA and DHA may be produced by transesterification or hydrolysis of a marine oil starting materials in order to give esters (typically ethyl esters) or free fatty acids or other derivatives that are suitable for further concentration of the omega-3 fatty acids. In some embodiments, the LC-PUFA esters are reesterified to a glyceride molecule to provide an oil with an increased concentration of omega-3 fatty acids. See, e.g., WO/2009/139641 and WO/2008/060163, the entire contents of which are incorporated herein by reference.

In some embodiments, the LC-PUFA derivative is an ester. Suitable esters include, but are not limited to, ethyl esters and methyl esters of LC-PUFAs. Preferred LC-PUFAs are listed above in the description for phospholipid compounds. Particularly preferred LC-PUFA esters include esters of EPA, DHA, and combination thereof. In some embodiments, compositions comprising the esters are utilized. In some embodiments, the compositions comprise an ester fraction comprising a mixture of two or more of the esters described above. In some embodiments, the fatty acid content of the ester fraction is from about 1% to about 99% LC-PUFA on a weight/weight basis (w/w; calculated as the weight of LC-PUFA in the ester fraction divided by the total weight of fatty acids in the ester fraction) or molar ratio basis (moles of LC-PUFA in the composition expressed as a percentage of the moles total fatty acids), 10% to 40% LC-PUFA w/w or molar ratio, 20% to 40% LC-PUFA w/w or molar ratio, 20% to 50% LC-PUFA w/w or molar ratio, 40% to 60% LC-PUFA w/w or molar ratio, 40% to 99% LC-PUFA w/w or molar ratio, 60% to 99% LC-PUFA w/w or molar ratio, or 80% to 99% LC-PUFA w/w or molar ratio. The w/w % may preferably be determined by an analytical method selected from the group consisting of gas chromatography (GC), high performance liquid chromatography (HPLC), GC-mass spectrometry (GC-MS), nuclear magnetic resonance (NMR) or other suitable methods as is known in the art. In some preferred embodiments, the LC-PUFA moieties in the esters are greater than 50%, 60%, 70%, 80%, 90% or 95% w/w EPA and/or DHA. In some embodiments, the ratio of EPA to DPA is from about 10:1 to 1:10, 3:1 to 1:3, 5:1 to 1:1, 2:1 to 1:1, 3:1 to 1:1, 1:1 to 1:3, or 1:1 to 1:5 on a molar basis. In some embodiments, the compositions comprise greater than about 40%, 50%, 60%, 70%, 80%, 90% or 95% esters w/w.

The ester compounds and compositions of the present invention may be provided from a variety of sources. In some embodiments, the esters compositions are prepared from a marine oil starting material. Suitable marine oils include, but are not limited to, krill oil, tuna oil, herring oil, menhaden oil, cod liver oil and algae oil. Methods for increasing the concentration of desirable omega-3 fatty acid moieties such as EPA and DHA in the esters are known in the art. See, e.g., WO/2009/139641, WO/2008/060163, and U.S. Pat. No. 5,656,667, the entire contents of each of which are incorporated herein by reference.

In some embodiments, the LC-PUFA is provided as a free fatty acid. Preferred LC-PUFAs are listed above in the description for phospholipid compounds. Particularly preferred LC-PUFAs include EPA, DHA, and combination thereof. In some embodiments, compositions comprising the free fatty acids are utilized. In some embodiments, the compositions comprise an free fatty acid fraction comprising a mixture of two or more of the free fatty acids described above. In some embodiments, the fatty acid content of the composition is from about 1% to about 99% LC-PUFA on a weight/weight basis (w/w; calculated as the weight of LC-PUFA in the composition divided by the total weight of fatty acids in the composition or molar ratio basis (moles of LC-PUFA in the composition expressed as a percentage of the moles total fatty acids), 10% to 40% LC-PUFA w/w or molar ratio, 20% to 40% LC-PUFA w/w or molar ratio, 20% to 50% LC-PUFA w/w or molar ratio, 40% to 60% LC-PUFA w/w or molar ratio, 40% to 99% LC-PUFA w/w or molar ratio, 60% to 99% LC-PUFA w/w or molar ratio, or 80% to 99% LC-PUFA w/w or molar ratio. The w/w % may preferably be determined by an analytical method selected from the group consisting of gas chromatography (GC), high performance liquid chromatography (HPLC), GC-mass spectrometry (GC-MS), nuclear magnetic resonance (NMR) or other suitable methods as is known in the art. In some preferred embodiments, the LC-PUFA content in the composition greater than 20%, 30%, 40%, 50%, 60%, 70%, 80%, 90% or 95% w/w EPA and/or DHA. In some embodiments, the ratio of EPA to DPA is from about 10:1 to 1:10, 3:1 to 1:3, 5:1 to 1:1, 3:1 to 1:1, 2:1 to 1:1, 1:1 to 1:3, or 1:1 to 1:5 on a molar basis. In some embodiments, the compositions comprise greater than about 40%, 50%, 60%, 70%, 80%, 90% or 95% esters w/w.

The free fatty acids and free fatty acid compositions of the present invention may be provided from a variety of sources. In some embodiments, the compositions are prepared from a marine oil starting material. Suitable marine oils include, but are not limited to, krill oil, tuna oil, herring oil, menhaden oil, cod liver oil and algae oil. Methods for increasing the concentration of desirable omega-3 fatty free fatty acids such as EPA and DHA are known in the art. See, e.g., WO/2009/139641, WO/2008/060163, and U.S. Pat. No. 5,656,667, the entire contents of each of which are incorporated herein by reference.

In some embodiments, the LC-PUFA compositions comprise a mixture of two or more of the phospholipid compounds or compositions, glyceride compounds or compositions, ester compounds or compositions or free fatty acids or free fatty acid compositions described above. For example, in some embodiments, the LC-PUFA composition may comprise from about 1% to about 60% phospholipids, with the remaining 99% to 40% of the composition being glycerides, esters, or free fatty acids or a combination thereof. In some embodiments, the LC-PUFA composition may comprise from about 10% to about 60% phospholipids, with the remaining 90% to 40% of the composition being glycerides, esters, or free fatty acids or a combination thereof. In some embodiments, the LC-PUFA composition may comprise from about 30% to about 60% phospholipids, with the remaining 70% to 40% of the composition being glycerides, esters, or free fatty acids or a combination thereof. In some preferred embodiments, the composition is a krill oil composition comprising from about 40% to 60% phospholipids and from about 20% to 45% glycerides.

In some embodiments, the compounds or compositions described above are administered to a subject in need thereof to treat a disease or condition associated with red blood cells and cell membranes, and in particular a disease or conditions associated with an abnormality in red blood cells of cell membranes. In some embodiments, the condition or disease is sickle cell disease, sickle cell anemia, or sickle cell trait. In some embodiments, the condition or disease is thalassemia (alpha-, beta- or delta-), thalassemia in combination with a hemoglobinopathy (Hemoglobin E, Hemoglobin S, or Hemoglobin C), splenomegaly, or membrane abnormities such as acanthocytes or spur/spike cells, codocytes (target cells), echinocytes (burr cells), elliptocytes and ovalocytes, spherocytes, stomatocytes (mouth cells) and degmacytes (“bite cells”).

In some embodiments, the subject is provided with a daily dosage comprising an effective amount of the compound or composition. In some embodiments, the amount is effective to prevent, alleviate or otherwise alter one or symptoms associated with the disease or condition. With respect to sickle cell disease, anemia, or trait the effective amount is sufficient to alleviate, prevent or alter one or more of the following symptoms: attacks of abdominal pain, bone pain, breathlessness, delayed growth and puberty, fatigue, fever, paleness, rapid heart rate, ulcers on the lower legs (in adolescents and adults), yellowing of the eyes and skin (jaundice), chest pain, excessive thirst, frequent urination, painful and prolonged erection (priapism—occurs in 10-40% of men with the disease), poor eyesight/blindness, strokes, and skin ulcers. In some embodiments, the daily dosage is effective to alleviate, prevent or alter one or more complication associated with sickle cell: acute chest syndrome, anemia, stroke, disease of many body systems such as kidney, liver, and lung, erectile dysfunction (as a result of priapism), gallstones, hemolytic crisis, cholecystitis, osteomyelitis urinary tract infection, joint destruction, ulcers, loss of function in the spleen, parvovirus B 19 infection, leading to low red blood cell production (aplastic crisis), splenic sequestration syndrome, and tissue death in the kidney. In some embodiments, the effective amount comprises from about 0.1 to about 5 grams of the LC-PUFA compound or composition, preferably from about 0.2 to about 3 grams of the LC-PUFA compound or composition, and most preferably about 0.5 to about 1.5 grams of the LC-PUFA compound or composition.

The LC-PUFA compounds and compositions of the present invention may be used to treat a variety of subjects. Suitable subjects include humans as well as domestic animals, non-human primates, and companion animals such as dogs, cats and birds. Due to the safety and tolerability of the LC-PUFA compounds and compositions, the compounds and compositions may be administered to children in need of treatment. In some preferred embodiments, treatment of sickle cell disease with the compounds and compositions of the present invention may commence at 1, 2, 3, 4, 5, 6, 7, 8, 9 or 10 years of age.

In some embodiments, the compounds or composition of the present invention are co-administered with one or more additional agents, preferably biologically active agents. In some embodiments, the agents are agents that are used to treat sickle cell disease or being developed to treat sickle cell disease. Such agents include, but are not limited to, hydroxyurea, chelators such as EDTA, antibiotics, pain relievers such as aspirin, NSAIDs, and morphine-based pain relievers, niprisan, piracetam, selectin inhibitors such as GMI-1070, ion channel blockers such as senicapoc, and DNA methyltransferase inhibitors such as decitabine.

The compounds and compositions of the present invention are preferably administered intravenously or orally. Accordingly, in some embodiments, the compositions of this invention (such as those described in the preceding sections) are contained in acceptable excipients and/or carriers for oral consumption or for intravenous administration. The actual form of the carrier, and thus, the composition itself, is not critical. The carrier may be a liquid, gel, gelcap, capsule, powder, solid tablet (coated or non-coated), tea, or the like. The composition is preferably in the form of a tablet or capsule and most preferably in the form of a soft gel capsule. Suitable excipient and/or carriers include vegetable oil, fish oil, krill oil, maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Maack Publishing Co., Easton, Pa.). For intravenous administration, the LC-PUFA compounds and compositions may preferably be provided as emulsions.

In some embodiments, the compounds and compositions are formulated for oral administration with flavoring agents or sweeteners. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. In addition to the carbohydrates described above, the nutritional supplement can contain natural or artificial (preferably low calorie) sweeteners, e.g., saccharides, cyclamates, aspartamine, aspartame, acesulfame K, and/or sorbitol.

The compositions of the present invention may also be delivered as dietary supplements, nutritional supplements, or functional foods.

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: asorbates (ascorbic acid, mineral ascorbate salts, rose hips, acerola, and the like), dehydroepiandosterone (DHEA), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In other embodiments, the present invention provides nutritional supplements (e.g., energy bars or meal replacement bars or beverages) comprising of the compositions of the present invention. In preferred embodiments, the nutritional supplements comprise an effective amount of the components as described above. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

In still further embodiments, the present invention provides food products, prepared food products, or foodstuffs (i.e., functional foods) comprising of the fatty acids or derivatives thereof. In preferred embodiments, the foods comprise an effective amount of the components as described above. For example, in some embodiments, beverages and solid or semi-solid foods comprising the fatty acids or derivatives thereof are provided. These forms can include, but are not limited to, beverages (e.g., soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

All publications and patents mentioned in the above specification are herein incorporated by reference. Various modifications and variations of the described compounds, compositions, methods and uses of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the medical, biological and chemical sciences are intended to be within the scope of the following claims.

### Example 1

Krill phospholipids were administered to 12 adults and 13 children with sickle cell disease on a daily basis. The patients were followed up with questionnaires pain episode recurrence and intensity as well as other parameters. Comparison of the results of the Patient Care Questionnaire for visits 1 and 2 shows that 12 participants experienced overall improvement in all indices; (6 adults and 6 children). Many participants experienced a decrease in pain episodes and intensity; for the participants whose health improved, all experienced a decrease in pain; and two did not need pain medication at all since first visit.

In a second study, krill phospholipids were administered to 16 adults and 9 children with sickle cell disease on a daily basis. Information was acquired during discussion with site staff. More than half of the participants indicated an improvement in their health after the enrolment visit. Comparison of the results of the Patient Care Questionnaire for visits 1 and 2 shows that 6 participants experienced overall improvement in all indices, (3 adults and 3 children); 2 did not experience any change (1 Adult and 1 child). The improvements included requiring less pain medication, fewer pain incidents, and looking and feeling better. Many participants experienced a decrease in pain episodes and intensity.

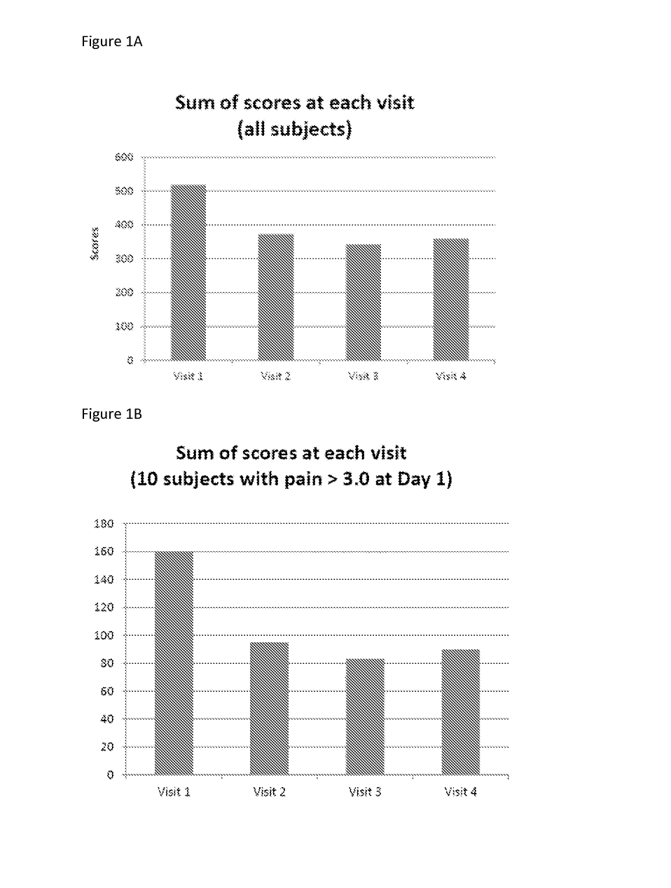

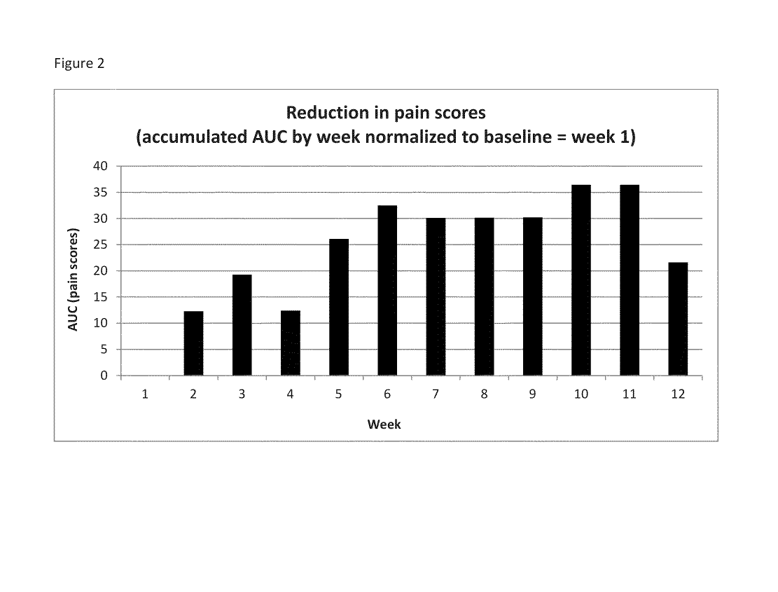

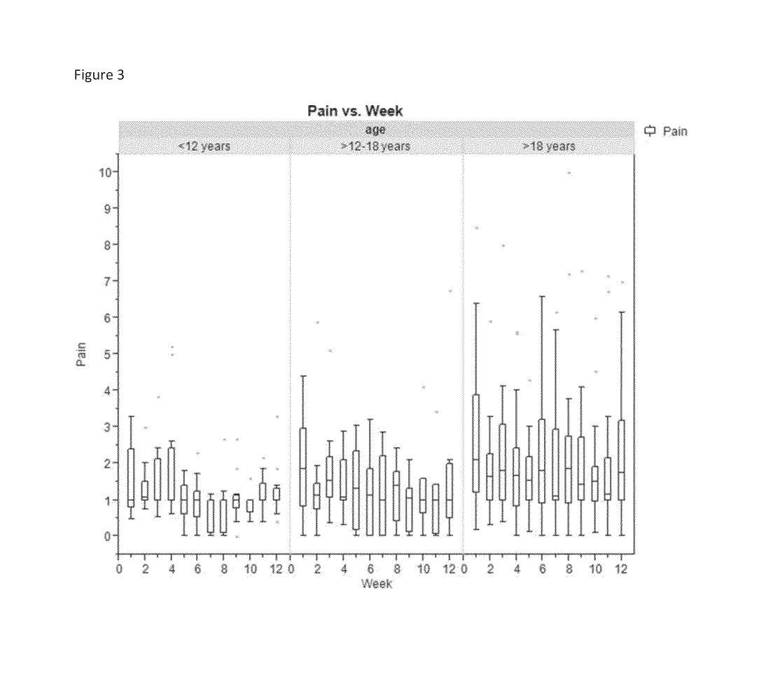

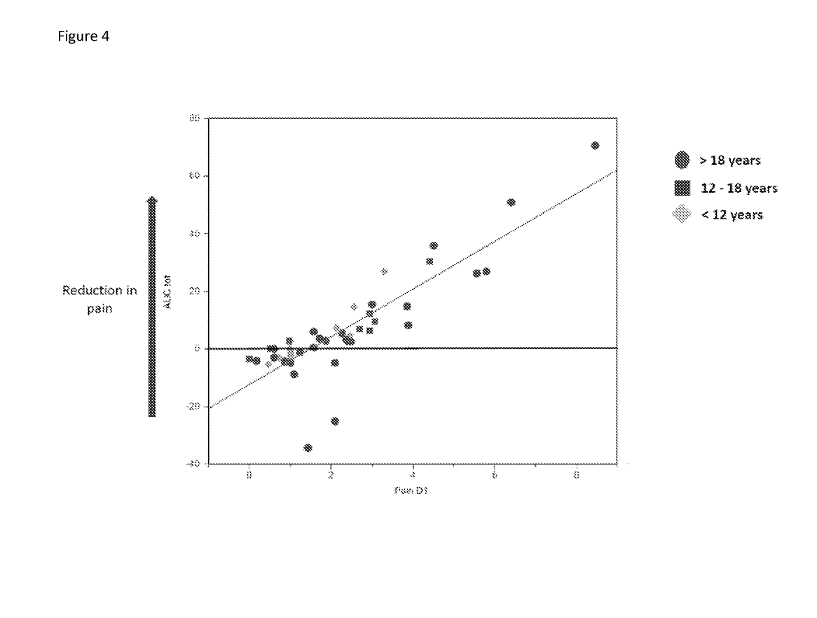

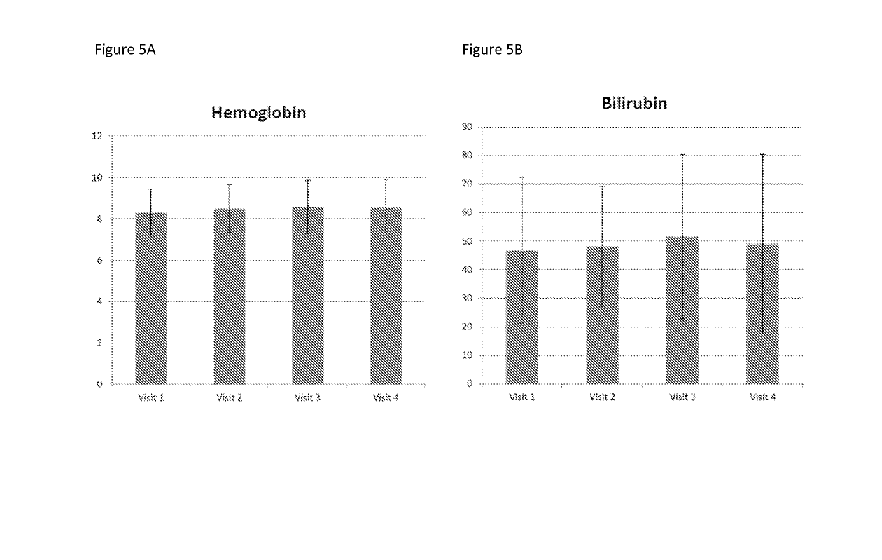

The data is presented in FIGS. 1-5. The results from this study show a) a trend of positive effects on quality of life measured by questionnaire (FIGS. 1a and b), b) the effect increases during a twelve week period (FIG. 2), c) variability is less in the under 12 age group (FIG. 3), d) when the pain score is high in week 1, the effect on pain reduction is greater (FIG. 4), and e) there are no objective changes in hemoglobin and bilirubin (FIG. 5). The questionnaire contained the following questions:

1) Is it hard for me to breathe?

2) Is it hard for me to walk?

3) Is it hard for me to run?

4) Is it hard for me to do sports activity or exercise?

5) I hurt or ache.

6) I have low energy.

7) I cannot do things other people can do.

8) I miss school or work because I do not feel well.

9) I miss school or work to go to the doctor or hospital.

The patients answered these questions with the following scale: 0—never; 1—Almost never; 2—Sometimes; 3—Often; 4—Almost always.

1. A method of treating a disease or condition associated with abnormal red blood cells or cell membranes in subject, improving the quality of life of a subject with sickle cell disease, or providing palliative care for a subject with sickle cell disease comprising:
administering to said subject a composition comprising at least one compound selected from the group consisting of a phospholipid compound: 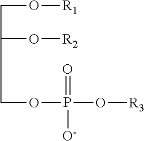
wherein at least one of R1 and R2 is a long chain polyunsaturated fatty acid moiety, the other of R1 and R2 is H or other organic moiety, and R3 is H or selected from a choline, ethanolamine, inositol and serine moiety,
a glyceride compound: 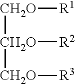
wherein at least one of either R1, R2, and R3 is a long chain polyunsaturated fatty acid moiety, and the other two of either R1, R2 and R3 may be H or a fatty acid moiety, including a long chain polyunsaturated fatty acid moiety,
esters and free fatty acids of long chain polyunsaturated fatty acids, and combinations thereof.

2. Method of claim 1, wherein said composition comprises at least 30% long chain polyunsaturated fatty acid moieties on a w/w basis.

3. Method of claim 1, wherein said composition comprises at least 40% long chain polyunsaturated fatty acid moieties on a w/w basis.

4. Method of claim 1, wherein said composition comprises at least 60% long chain polyunsaturated fatty acid moieties on a w/w basis.

5. Method of claim 1, wherein said composition comprises at least 80% long chain polyunsaturated fatty acid moieties on a w/w basis.

6. Method of claim 1, wherein said long chain fatty acid moieties are selected from the group consisting of eicosapentaenoic acid, docosahexaenoic acid, and combinations thereof.

7. Method of claim 1, wherein said composition comprises at least 40% w/w of said phospholipid compounds.

8. Method of claim 1, wherein said composition comprises at least 60% w/w of said phospholipid compounds.

9. Method of claim 1, wherein said composition comprises at least 80% w/w of said phospholipid compounds.

10. Method of claim 7, wherein said composition comprises a mixture of said phospholipid compounds, said mixture comprising at least 20% w/w long chain polyunsaturated fatty acid moieties.

11. Method of claim 7, wherein said composition comprises a mixture of said phospholipid compounds, said mixture comprising at least 30% w/w long chain polyunsaturated fatty acid moieties.

12. Method of claim 7, wherein said composition comprises a mixture of said phospholipid compounds, said mixture comprising at least 50% w/w long chain polyunsaturated fatty acid moieties.

13. Method of claim 10, wherein said long chain polyunsaturated fatty acid moieties are selected from the group consisting of eicosapentaenoic acid, docosahexaenoic acid, and combinations thereof.

14. Method of claim 13, wherein greater than 90% w/w of said long chain polyunsaturated fatty acid moieties are selected from the group consisting of eicosapentaenoic acid, docosahexaenoic acid, and combinations thereof are bound at position R2 of said phospholipid compound.

15. Method of claim 13, herein said eicosapentaenoic acid and said docosahexaenoic acid are present in a ratio of eicosapentaenoic acid:docosahexaenoic acid of from about 1:1 to about 3:1.

16. Method of claim 7, wherein said composition comprises a mixture of said phospholipid compounds, said mixture comprising at least 80% of said phospholipid compounds having a choline moiety at position R3.

17. Method of claim 7, wherein said composition comprises a mixture of said phospholipid compounds, said mixture comprising at least 90% of said phospholipid compounds having a choline moiety at position R3.

18. Method of claim 7, wherein said composition comprises at least 10% ethyl esters of said long chain polyunsaturated fatty acids.

19. Method of claim 7, wherein said composition comprises at least 30% ethyl esters of said long chain polyunsaturated fatty acids.

20. Method of claim 7, wherein said composition comprises at least 50% ethyl esters of said long chain polyunsaturated fatty acids.

21. Method of claim 18, wherein said esters of long chain polyunsaturated fatty acid comprise a long chain polyunsaturated fatty acid moiety selected from the group consisting of eicosapentaenoic acid, docosahexaenoic acid, and combinations thereof.

22. Method of claim 7, wherein said composition comprises a mixture of said glyceride compounds, said mixture comprising at least 30% w/w long chain polyunsaturated fatty acid moieties.

23. Method of claim 7, wherein said composition comprises a mixture of said glyceride compounds, said mixture comprising at least 40% w/w long chain polyunsaturated fatty acid moieties.

24. Method of claim 7, wherein said composition comprises a mixture of said glyceride compounds, said mixture comprising at least 50% w/w long chain polyunsaturated fatty acid moieties.

25. Method of claim 22, wherein said long chain polyunsaturated fatty acid moieties are selected from the group consisting of eicosapentaenoic acid, docosahexaenoic acid, and combinations thereof.

26. Method of claim 1, wherein said composition comprises astaxanthin.

27. Method of claim 1, wherein said composition comprises at least a second antioxidant.

28. Method of claim 1, wherein said composition is partially or totally derived from krill.

29. Method of claim 1, wherein said composition is administered in a formulation selected from the group consisting of a capsule, a tablet, a liquid, a powder, an emulsion, a dietary supplement, a nutritional supplement, a beverage and a functional food.

30. Method of claim 1, wherein said composition is administered by a route selected from the group consisting of oral administration and intravenous administration.

31. Method of claim 1, wherein said composition is administered in a daily dose of from about 0.1 to about 3 grams.

32. Method of claim 1, wherein said composition is administered to a subject selected from the group consisting of humans, non-human primates, domestic raised or farmed animals, and companion animals.

33. Method of claim 1, wherein said disease or condition associated with abnormal red blood cells or cell membranes is selected from the group consisting of sickle cell anemia, sickle cell disease, sickle cell trait, thalassemia, hemaglobinopathies, splenomegaly, presence of acanthocytes, presence of codocytes, presence of echinocytes, presence of burr cells, presence of elliptocytes, presence of ovalocytes, presence of spherocytes, presence of stomatocytes, presence of degmacytes, and combinations thereof.

34. Method of claim 1, wherein a second active agent is coadministered with said composition.

35. Method of claim 34, wherein said second active agent is selected from the group consisting of hydroxyurea, chelators, antibiotics, pain relievers, NSAIDs, niprisan, piracetam, selectin inhibitors, ion channel blockers, and DNA methyltransferase inhibitors.

36. Method of claim 1, wherein said subject is a juvenile subject of from 2 to 18 years in age.

37. Method of claim 1, wherein said composition is administered in a daily dosage sufficient to provide relief from symptoms associated with sickle cell disease in within two weeks from first administration of the composition.

38. Method of claim 37, wherein said symptoms are selected from the group consisting of attacks of abdominal pain, bone pain, breathlessness, delayed growth and puberty, fatigue, fever, paleness, rapid heart rate, ulcers on the lower legs, yellowing of the eyes and skin, chest pain, excessive thirst, frequent urination, painful and prolonged erection, poor eyesight/blindness, strokes, skin ulcers, acute chest syndrome, anemia, stroke, gallstones, hemolytic crisis, cholecystitis, osteomyelitis urinary tract infection, joint destruction, loss of function in the spleen, splenic sequestration syndrome, and tissue death in the kidney.

39. Method of claim 1, wherein said relief from said symptoms is independent of a reduction in bilirubin.

40. A method of treating a disease or condition associated with abnormal red blood cells or cell membranes in subject, improving the quality of life of a subject with sickle cell disease, or providing palliative care for a subject with sickle cell disease comprising: administering to said subject a composition comprising krill oil, said krill oil comprising from about 40% to about 60% w/w phospholipids and from about 100 mg/kg astaxanthin to about 2500 mg/kg astaxanthin.
